(19) United States
(12) Patent Application Publication
(10) Pub. No.: US 2008/0125705 A1
(43) Pub. Date: May 29, 2008

(54) Therapeutic treatment device for body lumen occlusion and method for occluding body lumen using the same

(75) Inventors: Taisuke Sato, Tokyo (JP); Masaya Yoshihara, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Medical Systems Corporation, Tokyo (JP)

(21) Appl. No.: 11/633,350

(22) Filed: Dec. 4, 2006

(30) Foreign Application Priority Data
Nov. 28, 2006 (JP) 2006-320693

(51) Int. Cl. A61M 25/00; A61N 1/00

(52) U.S. Cl. 604/93.01; 606/191; 607/2

(57) ABSTRACT

A therapeutic treatment device for body lumen occlusion of the invention includes: a pliable tubular main body portion including a fluid path inside thereof, the pliable tubular main body portion being inserted into a body lumen; an energy generating portion disposed on a side of one end of the main body portion; and an aperture portion communicating with the fluid path, the aperture portion being disposed in proximity to the energy generating portion. This allows the present invention to achieve a therapeutic treatment device for body lumen occlusion inexpensively produceable with a simple constitution, which can prevent damaging a body lumen and selectively occlude the body lumen.

This application claims benefit of Japanese Application No. 2006-320693 filed on Nov. 28, 2006, the contents of which are incorporated by this reference.

### BACKGROUND OF THE INVENTION

1. Field of the Invention

The invention relates to a therapeutic treatment device for body lumen occlusion, and a method for occluding a body lumen using the therapeutic treatment device for body lumen occlusion.

2. Description of Related Art

Conventionally, there have been catheters which are inserted into a body lumen, especially a blood vessel, to perform a predetermined treatment.

In one exemplary case, such catheters are used for lower-limb varices in which leg blood vessels swell like weals or small blood vessels distend in a sanguine mesh-like shape on the back of a femur or genu.

The lower-limb varices are a disease in which lower-limp surface veins swell like phymas, and are caused mainly by blood backflow due to vein valve destruction. The lower-limb varices, left untreated, will gradually progress causing symptoms stemming from vein varices or lower-limb vein congestion, which is concerned to lead to a complication accompanying them.

As a treatment tool for selectively occluding a blood vessel which is a body lumen for treating the lower-limb varices, e.g., PCT Japanese Translation Patent Publication No. 11-506651 discloses a method and a device for permanent vessel occlusion.

The method and device for permanent vessel occlusion as described in the PCT Japanese Translation Patent Publication No. 11-506651 permanently occlude a part of a blood vessel by physically collapsing the blood vessel and subsequently applying a high-frequency to the blood vessel for thermal coagulation thereof.

This publication discloses, before the high-frequency application, adhering a part of the blood vessel by flatly pushing and spreading the part in an outer radial direction from inside the vessel, or adhering the blood vessel by spreading a pincette-like jaw portion inside the vessel to hook and thereafter grasp the vessel, and further, e.g., adhering the blood vessel by expanding a balloon in the blood vessel to stop the blood flow and then negatively pressurizing inside the vessel.

### SUMMARY OF THE INVENTION

A therapeutic treatment device for body lumen occlusion of the invention includes: a pliable tubular main body portion including a fluid path inside thereof, the pliable tubular main body portion being inserted into a body lumen; an energy generating portion disposed on a side of one end of the main body portion; and an aperture portion communicating with the fluid path, the aperture portion being disposed in proximity to the energy generating portion. This allows the present invention to achieve a therapeutic treatment device for body lumen occlusion which prevents damaging a body lumen and which is inexpensively produceable with a simple constitution capable of selectively occluding the body lumen, and a method for occluding the body lumen using this therapeutic treatment device for body lumen occlusion.

The above and other objects, features and advantages of the invention will become more clearly understood from the following description referring to the accompanying drawings.

### BRIEF DESCRIPTION OF THE DRAWINGS

FIG. 1 is a view showing a configuration of a body-lumen-shrinking treatment system according to a first embodiment of the present invention.

FIG. 2 is a perspective view showing a distal end portion of a therapeutic treatment device for body lumen occlusion according to the first embodiment of the present invention.

FIG. 3 is a sectional view showing the distal end portion of the therapeutic treatment device for body lumen occlusion of FIG. 2, according to the first embodiment of the present invention.

FIG. 4 is a view showing a state where the therapeutic treatment device for body lumen occlusion is inserted into a vein, according to the first embodiment of the present invention.

FIG. 5 is a view showing a state of the vein when the therapeutic treatment device for body lumen occlusion has performed a suction from the state shown in FIG. 4, according to the first embodiment of the present invention.

FIG. 6 is a view showing a state where the vein is occluded by the therapeutic treatment device for body lumen occlusion, according to the first embodiment of the present invention.

FIG. 7 is a view showing a state where the vein is occluded, according to the first embodiment of the present invention.

FIG. 8 is a perspective view showing a distal end portion of a therapeutic treatment device for body lumen occlusion according to a second embodiment of the present invention.

FIG. 9 is a sectional view showing the distal end portion of the therapeutic treatment device for body lumen occlusion of FIG. 8, according to the second embodiment of the present invention.

FIG. 10 is a sectional view taken along the line X-X of FIG. 9, according to the second embodiment of the present invention.

FIG. 11 is a sectional view taken along the line XI-XI of FIG. 9, according to the second embodiment of the present invention.

FIG. 12 is a sectional view taken along the line XII-XII of FIG. 9, according to the second embodiment of the present invention.

FIG. 13 is a view showing a state where the therapeutic treatment device for body lumen occlusion is inserted into a vein, according to the second embodiment of the present invention.

FIG. 14 is a view showing a state of the vein when the therapeutic treatment device for body lumen occlusion has performed a suction from the state shown in FIG. 13, according to the second embodiment of the present invention.

FIG. 15 is a view showing a state where the vein is occluded by the therapeutic treatment device for body lumen occlusion, according to the second embodiment of the present invention.

FIG. 16 is a view showing a state where the vein is occluded, according to the second embodiment of the present invention.

FIG. 17 is a view showing a configuration of a body-lumen-shrinking treatment system according to a third embodiment of the present invention.

FIG. 18 is a perspective view showing a distal end portion of a therapeutic treatment device for body lumen occlusion according to the third embodiment of the present invention.

FIG. 19 is a sectional view showing the distal end portion of the therapeutic treatment device for body lumen occlusion of FIG. 18, according to the third embodiment of the present invention.

FIG. 20 is a view showing a state where the therapeutic treatment device for body lumen occlusion is inserted into the vein and a suction is performed, according to the third embodiment of the present invention.

FIG. 21 is a view showing a state where the vein is occluded by the therapeutic treatment device for body lumen occlusion, according to the third embodiment of the present invention.

FIG. 22 is a view showing a configuration of a body-lumen-shrinking treatment system according to a fourth embodiment of the present invention.

FIG. 23 is a view showing a state where a therapeutic treatment device for body lumen occlusion is inserted into a vein and an indocyanine green (ICG) solution is diffused therein, according to the fourth embodiment of the present invention.

FIG. 24 is a view showing a state where the indocyanine green (ICG) solution diffused in the vein is irradiated with laser light, according to the fourth embodiment of the present invention.

FIG. 25 is a view showing a state where the vein is occluded by the therapeutic treatment device for body lumen occlusion, according to the fourth embodiment of the present invention.

### DETAILED DESCRIPTION OF PREFERRED EMBODIMENTS

With reference to the drawings, embodiments of the present invention will be described below.

### First Embodiment

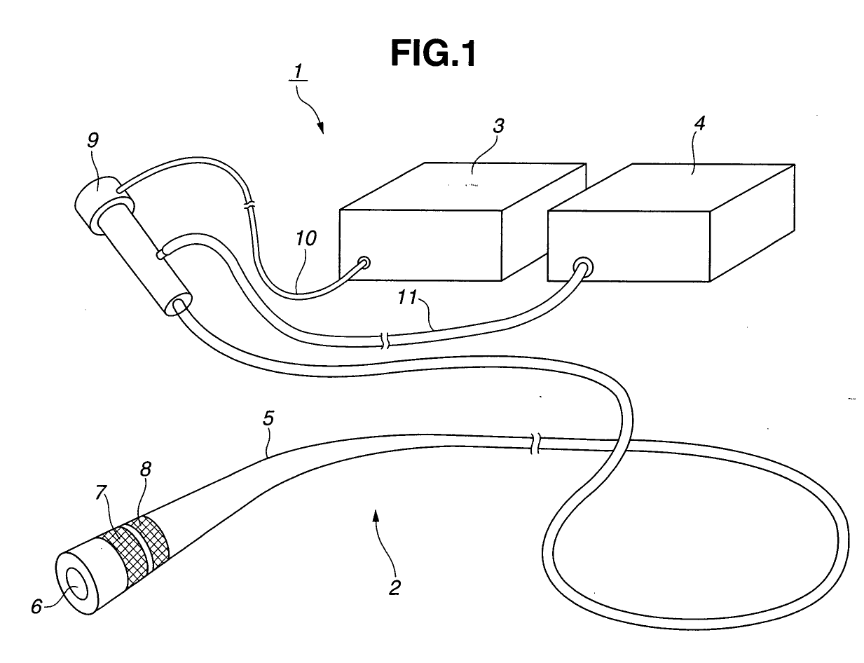

First, a first embodiment will be described using FIGS. 1 to 7.

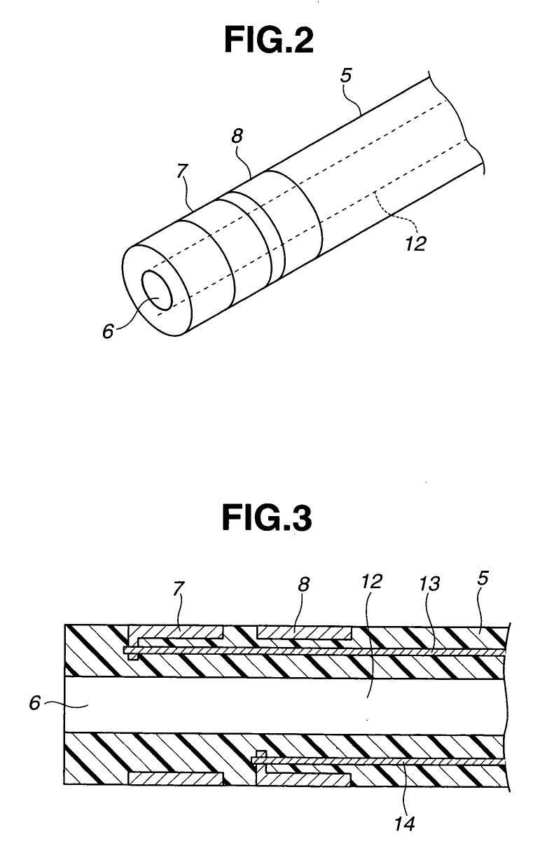

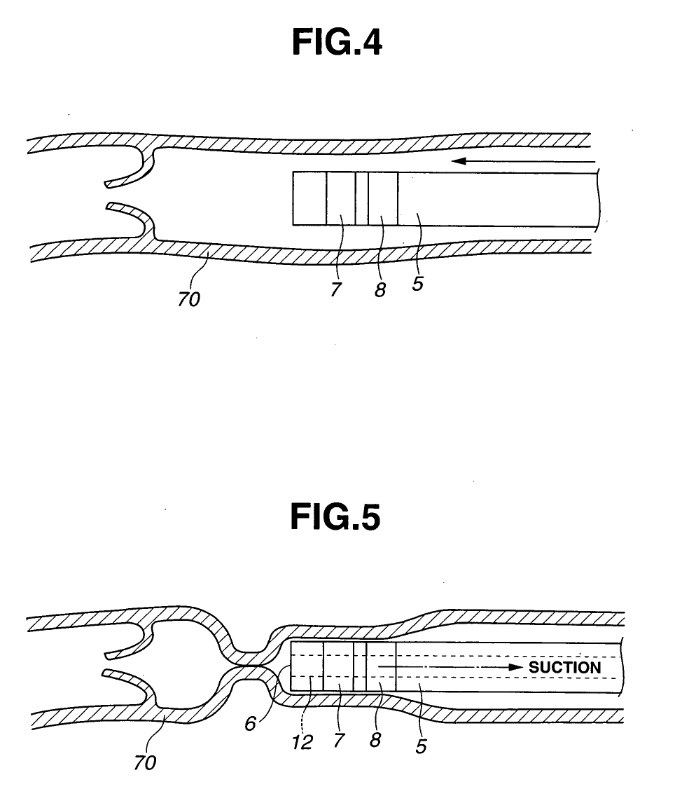

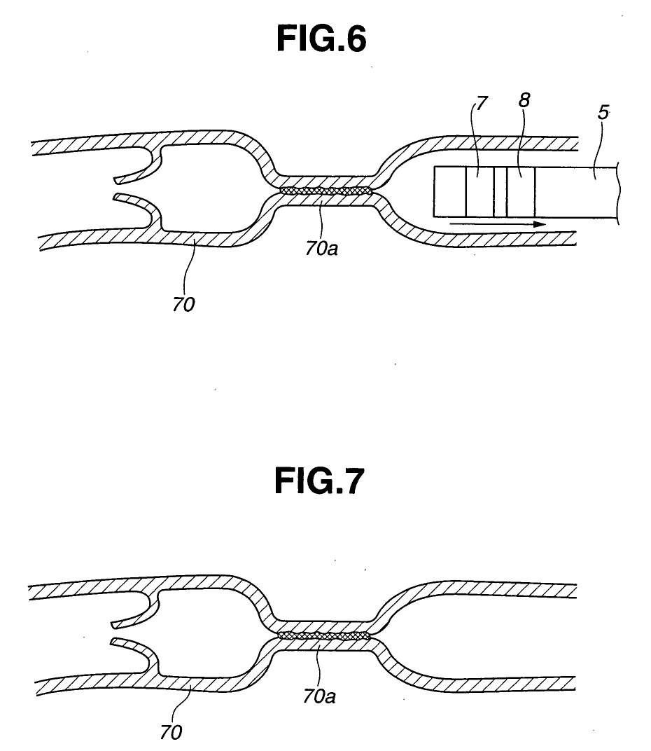

FIGS. 1 to 7 relate to the first embodiment of the present invention, wherein: FIG. 1 is a view showing a configuration of a body-lumen-shrinking treatment system; FIG. 2 is a perspective view showing a distal end portion of a therapeutic treatment device for body lumen occlusion; FIG. 3 is a sectional view showing the distal end portion of the therapeutic treatment device for body lumen occlusion of FIG. 2; FIG. 4 is a view showing a state where the therapeutic treatment device for body lumen occlusion is inserted into a vein; FIG. 5 is a view showing a state of the vein when the therapeutic treatment device for body lumen occlusion has performed suction from the state shown in FIG. 4; FIG. 6 is a view showing a state where the vein is occluded by the therapeutic treatment device for body lumen occlusion; and FIG. 7 is a view showing a state where the vein is occluded.

As shown in FIG. 1, a body-lumen-shrinking treatment system (also referred to as “vein-shrinking treatment system”) 1 includes a catheter 2 which is the therapeutic treatment device for body lumen occlusion, a high-frequency generating device 3 which is a high-frequency power source, and a suction device 4 which is a negative pressure generating device.

The catheter 2 includes a catheter main body portion 5 in a flexible tube shape, a suction opening 6 which is an aperture open at a distal end surface of the catheter main body portion 5, electrodes 7, 8 which are energy generation portions parallelly provided on a distal end outer circumference of the catheter main body portion 5, and a base portion 9 disposed at a proximal end of the catheter main body portion 5.

The base portion 9 of the catheter 2 is provided with a high-frequency connection port. The high-frequency connection port is electrically connected with one end of a cable 10, the other end of the cable 10 being detachably connected to the high-frequency generating device 3. The base portion 9 also has a suction connection port. The suction connection port is connected with one end of a suction tube 11 to communicate with a lumen 12 to be described later (see FIGS. 2 and 3) disposed in the catheter main body portion 5. The other end of the suction tube 11 is detachably connected to the suction device 4.

As shown in FIG. 2, in the catheter main body portion 5 are disposed the lumen 12 having the suction opening 6, which is an aperture portion, at a distal end surface of main body portion 5 serving as a suction port, and two lead lines 13, 14 having distal end portions connected to the two electrodes 7, 8, respectively.

Also, as shown in FIG. 3, the two electrodes 7, 8 of the present embodiment are attached and fitted to an outer circumference of the catheter main body portion 5. The electrodes 7, 8 are constituted by bipolar-type high-frequency electrodes on active and return sides, respectively. These two electrodes 7, 8 each have an outer circumferential surface as a respective surface which is steplessly provided along the entire outer circumferential surface of the catheter main body portion 5. Note that the high-frequency electrodes are not limited to the bipolar-type and may be of a monopolar-type.

The lead lines 13, 14 connected to the electrodes 7, 8 are inserted through the catheter main body portion 5 in a manner of being embedded therein to be isolated from each other herein, up to a high-frequency connection terminal (not shown) which is a high-frequency power source connection port provided to the base portion.

That is, the lead line 13 electrically transmits a high-frequency from the high-frequency generating device 3 to the electrode 7 via the above-mentioned cable 10 connected to the high-frequency connection terminal of the base portion 9. Then, the lead line 14 electrically transmits the high-frequency externally discharged from the electrode 7 to the electrode 8, to the high-frequency generating device 3 via the above-mentioned cable 10.

Note that the lead lines 13, 14 may be bare wires or electrical communication lines with insulating coating, so long as the lines are constituted to sufficiently maintain insulation from each other. Further, the high-frequency generating device 3 and the suction device 4 can perform desired operations using a foot switch or a controller unit which are not shown.

Now, referring to FIGS. 4 to 7, there will be described steps of conducting therapy for lower-limb varices by using the body-lumen-shrinking treatment system 1 of the present embodiment constituted as mentioned above.

First, a surgeon as a user prepares the body-lumen-shrinking treatment system 1 constituted by connecting the high-frequency generating device 3 and the suction device 4 to the catheter 2 as shown in FIG. 1. Then, the surgeon performs sectioning or the like, from skin side, of a part of the vein of a patient, which is a body lumen diseased by lower-limb varices, and inserts into the vein the catheter main body portion 5 of the catheter 2 from a distal end portion thereof.

The surgeon inserts and moves the distal end portion of the catheter main body portion 5 toward a position where the vein 70 is to be shrunk and coagulated, as shown in FIG. 4. Then, the surgeon drives the suction device 4 by operating the foot switch or the controller unit.

This results in that a suction force from the suction opening 6 (see FIG. 2) of the catheter main body portion 5 negatively pressurizes to thereby shrink a vessel wall of the vein 70 near the suction opening 6, as shown in FIG. 5. At this time, the vein 70 around the distal end portion is approximated to or finally adhered to the catheter main body portion 5.

Next, the surgeon drives the high-frequency generating device 3 by operating the foot switch or the controller unit. This causes a high-frequency current outputted from the high-frequency generating device 3 to be discharged from the active electrode 7.

The high-frequency current outputted from the active electrode 7 flows through a side surface at the distal end portion of the catheter main body portion 5 to be passed to the return electrode 8 while electrically conducting in the vessel wall of the vein 70 in proximity or adhesion to the electrodes 7, 8. At this time, at the vessel wall of the vein 70 in proximity or adhesion to the electrodes 7, 8, Joule heat is generated due to the high-frequency current, making the vessel wall shrink and coagulate.

Then, the surgeon stops the high-frequency generating device 3 by controlling the foot switch or the controller unit, and moves the catheter main body portion 5 in extracting direction thereof. Thereafter, there still exists a shrinking action continuing to work at the portion of the vessel wall of the vein 70 that received the Joule heat as shown in FIG. 6, which consequently shrinks and coagulates to thereby completely occlude the portion of the vessel wall of the vein 70 (a portion to be treated 70a), as shown in FIG. 7.

Note that driving the high-frequency generating device 3 without driving the suction device 4 will result in insufficient shrinkage and coagulation of the vein 70, because the vessel wall of the vein 70 is prevented from being approximated or adhered to the distal end portion of the catheter main body portion 5, causing dispersion of the generated Joule heat due to blood flow in the vein 70, thus inhibiting sufficient application of the Joule heat to the vessel wall of the vein 70.

As described above, with the body-lumen-shrinking treatment system 1 of the present embodiment, a blood vessel to be treated which is a lumen can be easily shrunk and coagulated and thus permanently occluded by the catheter 2 without damaging the blood vessel with an undue load. Moreover, the body-lumen-shrinking treatment system 1 greatly contributes to reducing the production defect rate with a simple constitution of the catheter 2 especially for treating the vessel 70 with a small diameter, whereby achieving inexpensive constitution with reduced production cost.

### Second Embodiment

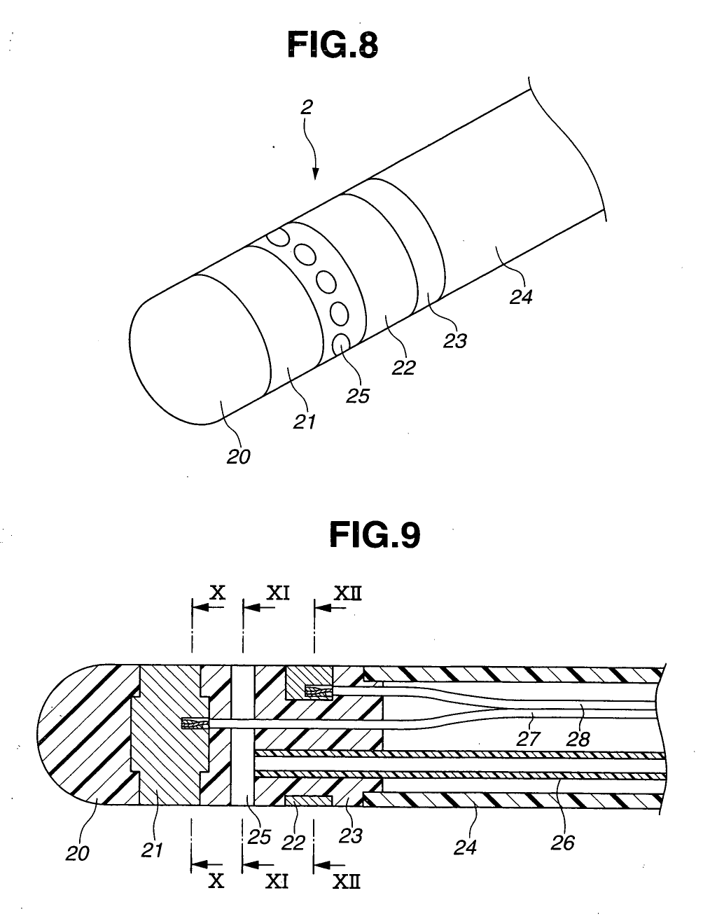

Next, a second embodiment of the present invention will be described using FIGS. 8 to 15. In the following description, the same components as in the first embodiment are attached with the same reference symbols while omitting descriptions thereof; only different parts will be described.

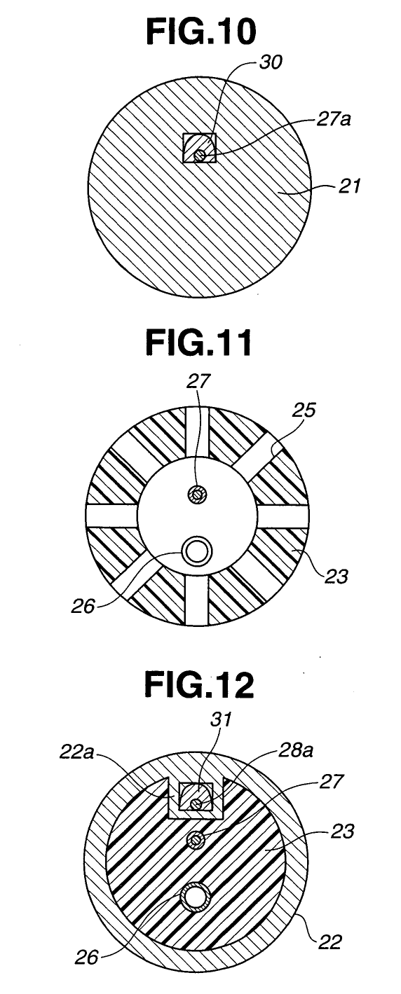

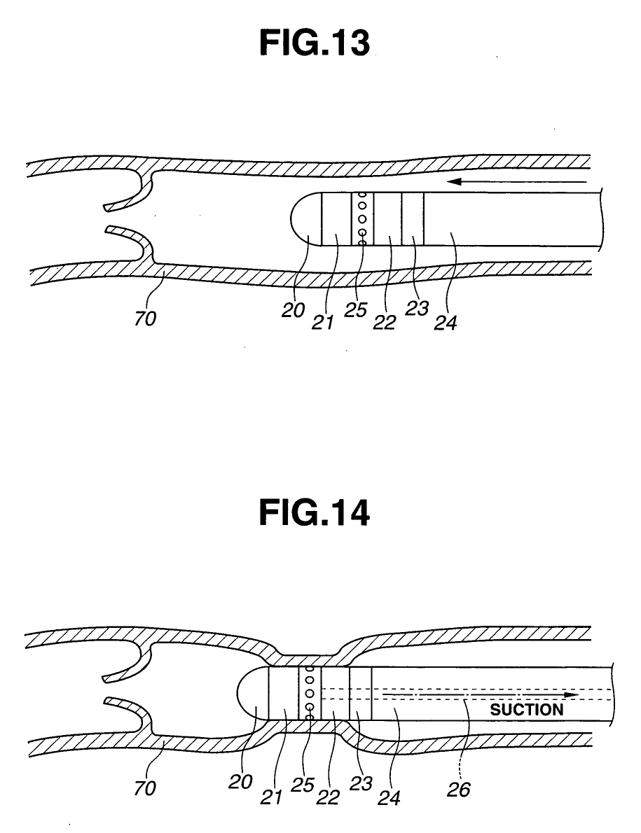

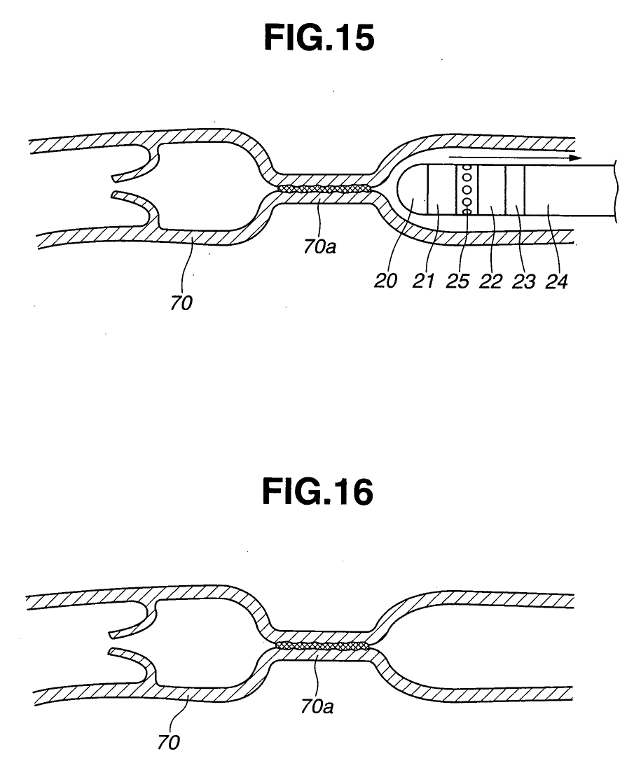

FIGS. 8 to 16 relate to the second embodiment of the present invention, wherein: FIG. 8 is a perspective view showing a distal end portion of a therapeutic treatment device for body lumen occlusion; FIG. 9 is a sectional view showing the distal end portion of the therapeutic treatment device for body lumen occlusion of FIG. 8; FIG. 10 is a sectional view taken along the line X-X of FIG. 9; FIG. 11 is a sectional view taken along the line XI-XI of FIG. 9; FIG. 12 is a sectional view taken along the line XII-XII of FIG. 9; FIG. 13 is a view showing a state where the therapeutic treatment device for body lumen occlusion is inserted into a vein; FIG. 14 is a view showing a state of the vein when the therapeutic treatment device for body lumen occlusion has performed suction from the state shown in FIG. 13; FIG. 15 is a view showing a state where the vein is occluded by the therapeutic treatment device for body lumen occlusion; and FIG. 16 is a view showing a state where the vein is occluded.

A catheter 2 of a body-lumen-shrinking treatment system 1 of this embodiment differs from that of the first embodiment mainly in the configuration of the distal end portion. Specifically, as shown in FIGS. 8 and 9, the catheter 2 includes a distal end flexible member 20 made of an insulative synthetic resin or the like, an active electrode 21, a return electrode 22, an insulation portion 23 made of a synthetic resin or the like, and a flexible tube 24, in this order from the distal end portion of the catheter 2.

The distal end flexible member 20 has a hemispherical dome shape in a distal end direction thereof to avoid damaging a lumen wall. The distal end flexible member 20 has a proximal end surface to which the active electrode 21 having a generally disc shape (see FIG. 10) is attached and fitted with adhesive or the like. The active electrode 21 has a proximal end surface to which the insulation portion 23 is attached and fitted with adhesive or the like.

The insulation portion 23 is attached and fitted with the return electrode 22 having a generally circular ring shape. The insulation portion 23 is a member having a generally cylindrical shape, and has, between the active electrode 21 and the return electrode 22, a plurality of, herein eight, suction ports 25 (see FIG. 11) as aperture portions formed radially toward an outer circumference orthogonal to the axial direction.

Inside the insulation portion 23, there is fixed a distal end portion of a tube 26 that constitutes a fluid path communicating to the suction ports 25. The tube 26 is inserted through the flexible tube 24 to the base portion 9, and communicates with the suction device 4 and the suction tube 11 shown in FIG. 1.

Note that the active electrode 21 and the return electrode 22 are electrically connected to lead lines 27, 28, respectively, which are inserted in the insulation portion 23. These lead lines 27, 28 are also inserted through the flexible tube 24 to the base portion 9, and are electrically connected to the high-frequency generating device 3 shown in FIG. 1 via the cable 10.

As is appreciated from the foregoing, the insulation portion 23 has hole portions through which to insert the tube 26 and the two lead lines 27, 28 as shown in FIG. 12. Note that also in this embodiment, the two electrodes 21, 22 each have an outer circumferential surface serving as a respective surface which is steplessly provided along the entire outer circumferential surface of the catheter main body portion 5.

The active electrode 21 has a hole-shaped connection portion to be connected, herein electrically by means of a solder 30, with a wire 27a of the lead line 27. In contrast, the return electrode 22 has a convex portion 22a oriented in an inner circumferential direction. In the convex portion 22a is formed a hole-shaped connection portion to which a wire of the lead line 28 is electrically connected by means of a solder 31. The respective connection between the electrodes 21, 22 and the lead lines 27, 28 is not limited to that using a solder, but may be connector-connection.

In the present embodiment, the catheter 2 only includes the flexible tube 24 which is flexible in the side toward the proximal end thereof from the distal end portion having the electrodes 21, 22, and the tube 26 and the lead lines 27, 28 which are inserted through the flexible tube 24 and are built-in objects therein. In other words, majority of the catheter 2 is formed by pliable and flexible parts and therefore is freely bendable. This permits the catheter 2 to freely advance and retract while flexing in a lumen such as a blood vessel.

Next, referring to FIGS. 13 to 15, there will be described steps of conducting therapy for lower-limb varices herein again as in the first embodiment by using the body-lumen-shrinking treatment system 1 of the present embodiment constituted as described above.

First, like in the first embodiment, the surgeon prepares the body-lumen-shrinking treatment system 1 constituted by connecting the high-frequency generating device 3 and the suction device 4 to the catheter 2, and subsequently inserts the catheter 2 from the side of the distal end flexible member 20 into a vein of a patient, which is a body lumen diseased by lower-limb varices.

The surgeon inserts and moves the distal end portion of the catheter 2 to a position where the vein 70 is to be shrunk and coagulated, as shown in FIG. 13. The surgeon drives the suction device 4 by operating the foot switch or the controller unit.

This results in that a suction force from each of the suction ports 25 negatively pressurizes to thereby shrink a vessel wall of the vein 70 near the suction ports 25, as shown in FIG. 14. At this time, the distal end portion of the catheter 2 is adhered with the vein 70 around the electrodes 21, 22.

In other words, in the present embodiment, a partial vessel wall of the vein 70 is absorbed to the suction ports 25 disposed between a pair of the electrodes 21, 22. That is, the partial vessel wall of the vein 70 thus sucked is in adhesion to respective surface of the electrodes 21, 22.

Next, the surgeon drives the high-frequency generating device 3 by controlling the foot switch or the controller unit. This causes a high-frequency current outputted from the high-frequency generating device 3 to be discharged from the active electrode 21 to the return electrode 22. At this time, the high-frequency current is passed to the return electrode 8 while electrically conducting to the vessel wall of the vein 70 in adhesion to the pair of electrodes 21, 22. Like in the first embodiment, at the vessel wall of the vein 70 in adhesion to each of the electrodes 21, 22, Joule heat is generated due to the high-frequency current, making the vessel wall shrink and coagulate.

Then, the surgeon stops the high-frequency generating device 3 by controlling the foot switch or the controller unit, and moves the catheter 2 in extracting direction thereof. Thereafter, like in the first embodiment, there still exists a shrinking action continuing to work at a portion of the vessel wall of the vein 70 which received the Joule heat as shown in FIG. 15, which consequently shrinks and coagulates to thereby completely occlude the portion of the vessel wall of the vein 70 (the portion to be treated 70a) as shown in FIG. 16.

As describe above, the catheter 2 of the body-lumen-shrinking treatment system 1 of this embodiment can not only provide the effects of the first embodiment, but also surely and efficiently transmit the Joule heat due to the high-frequency current discharged between the electrodes 21, 22 so as to thereby surely shrink and coagulate to occlude the lumen vessel, by radially forming a plurality of suction ports 25 which are open in the circumferential direction on the outer surface of the catheter between the pair of electrodes 21, 22, to absorb the vessel wall of the blood vessel as a lumen.

Note that, in the present embodiment, apertures of the plurality of suction ports 25 are provided between the pair of electrodes 21, 22. Although this arrangement can efficiently absorb the vessel wall near the apertures, no limitation is placed thereon. The apertures may be arranged on a distal end side of the active electrode 21 and/or a proximal end side of the return electrode 22, as long as these sides are in proximity to the electrodes 21, 22.

### Third Embodiment

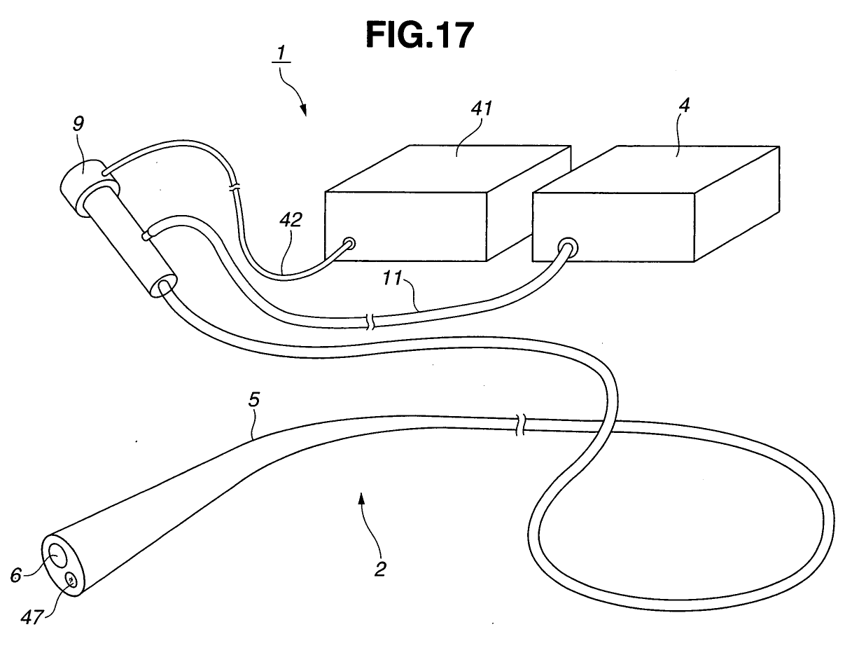

Next, a third embodiment of the present invention will be described using FIGS. 17 to 21. Also in the following description, the same components as in the first embodiment are attached with the same reference symbols while omitting descriptions thereof; only different parts will be described.

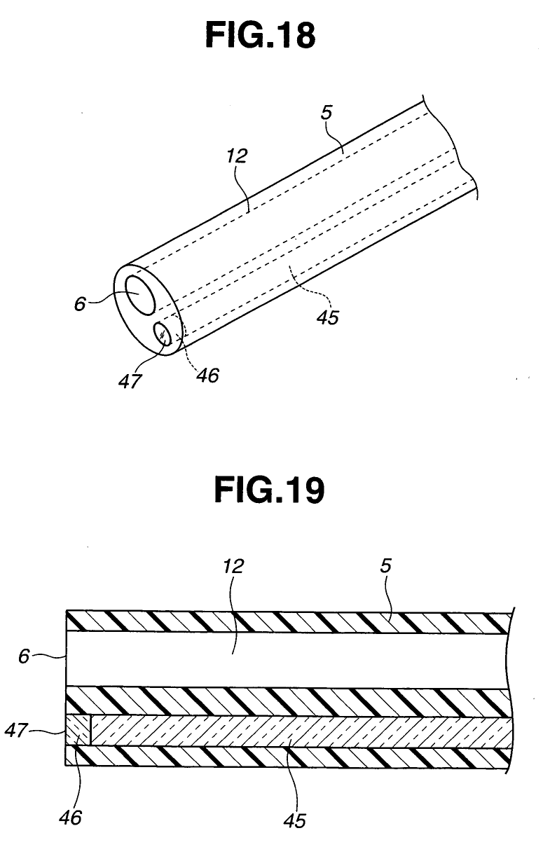

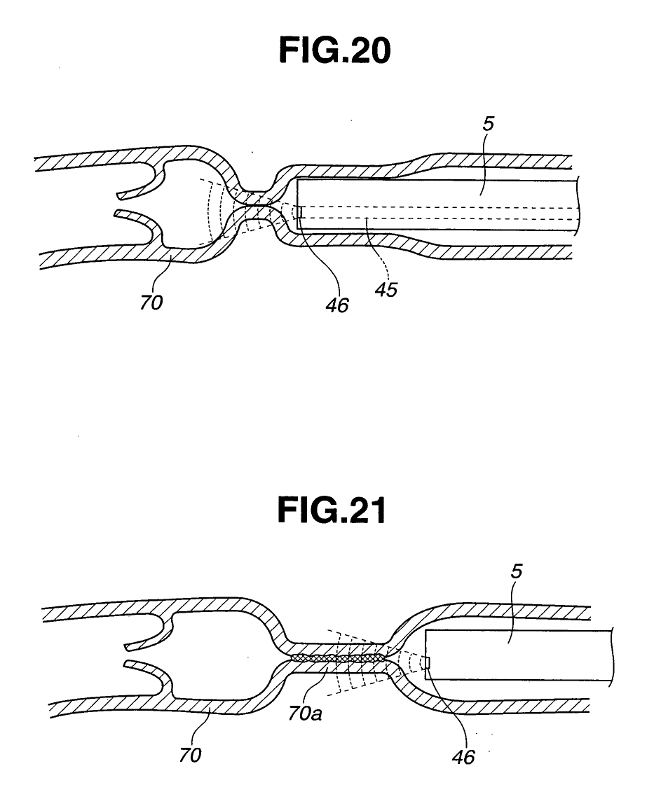

FIGS. 17 to 21 relate to the third embodiment of the present invention, wherein: FIG. 17 is a view showing a configuration of a body-lumen-shrinking treatment system; FIG. 18 is a perspective view showing a distal end portion of a therapeutic treatment device for body lumen occlusion; FIG. 19 is a sectional view showing the distal end portion of the therapeutic treatment device for body lumen occlusion of FIG. 18; FIG. 20 is a view showing a state where the therapeutic treatment device for body lumen occlusion is inserted into the vein and suction is performed; and FIG. 21 is a view showing a state where the vein is occluded by the therapeutic treatment device for body lumen occlusion.

A body-lumen-shrinking treatment system 1 of this embodiment includes an energy generating portion in a form different from the above-described embodiments. As shown in FIG. 17, the body-lumen-shrinking treatment system 1 of this embodiment uses a laser generating device 41 which is detachably connected to an optical fiber connection port at the base portion 9 of the catheter 2 via an optical cable 42, in place of the high-frequency generating device 3 of the first and second embodiments.

In line therewith, the catheter 2 includes an optical fiber 45 for transmitting laser light from the laser generating device 41, disposed in the catheter main body portion 5 and the base portion 9 (not shown), as shown in FIGS. 18 and 19. At a distal end of the catheter main body portion 5 is provided an irradiation lens 46 arranged to contact a distal end surface of the optical fiber 45.

The irradiation lens 46 is an optical part serving as an energy generating portion constituting an irradiation portion for forwardly irradiating the laser transmitted by the optical fiber 45 from a distal end surface 47 at an angle of 30 degrees, for example.

Next, referring to FIGS. 20 and 21, there will be described steps of conducting therapy for lower-limb varices herein again as in the afore-described embodiments by using the body-lumen-shrinking treatment system 1 of the present embodiment constituted as described above.

The surgeon inserts into the vein 70 the catheter main body portion 5 of the catheter 2 from the distal end portion thereof, and inserts and moves the distal end portion of the catheter main body portion 5 to a position where the vein 70 is to be shrunk and coagulated, as in the first embodiment.

Then, the surgeon drives the suction device 4 by operating the foot switch or the controller unit. This results in that a suction force from the suction opening 6 (see FIG. 2) causes the vein 70 around the distal end portion of the catheter main body portion 5 to be approximated to or finally adhered thereto, as shown in FIG. 20.

Next, the surgeon drives the laser generating device 41 by operating the foot switch or the controller unit. This causes laser light outputted from the laser generating device 41 to be irradiated from the irradiation lens 46 to a part of the vein 70 in proximity or adhesion to the distal end portion, via the optical fiber 45. As a result, the laser light generates heat on the vessel wall of the vein 70, and the heat thus generated shrinks and coagulates the vein wall. Note that, at this time, the surgeon is advised to irradiate the laser light to the portion to be treated 70a of the vein 70 while slightly moving the catheter main body portion 5 in extracting direction thereof.

Then, the surgeon stops the laser generating device 41 by operating the foot switch or the controller unit, and moves the catheter main body portion 5 in the extracting direction thereof.

Herein also, the portion to be treated 70a of the vein 70 which received the heat has shrinking action still continuing to exist, which shrinks and coagulates to thereby completely occlude the portion to be treated 70a, as shown in FIG. 21.

As described above, the body-lumen-shrinking treatment system 1 of the present embodiment can completely occlude the vein 70 which is a lumen, by means of the constitution including the laser generating device 41 for generating laser light, and the catheter 2 provided with the optical fiber 45 and the irradiation lens 46 for irradiating the laser light to the portion to be treated 70a. Accordingly, the body-lumen-shrinking treatment system 1 of this embodiment also provides the same effects as those in the first embodiment.

### Fourth Embodiment

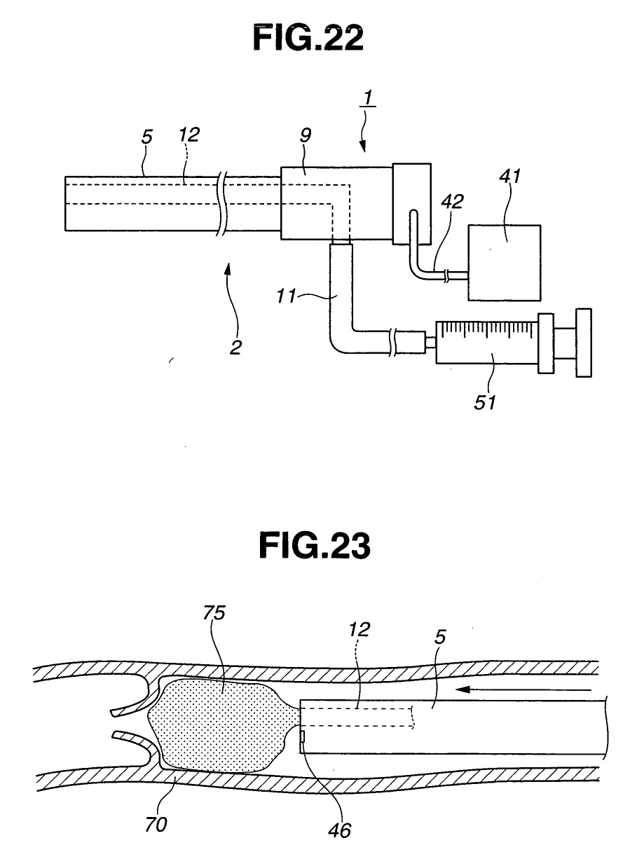

Lastly, a fourth embodiment of the present invention will be described using FIGS. 22 to 25. Also in the following description, the same components as in the first and third embodiments are attached with the same reference symbols while omitting descriptions thereof; only different parts will be described.

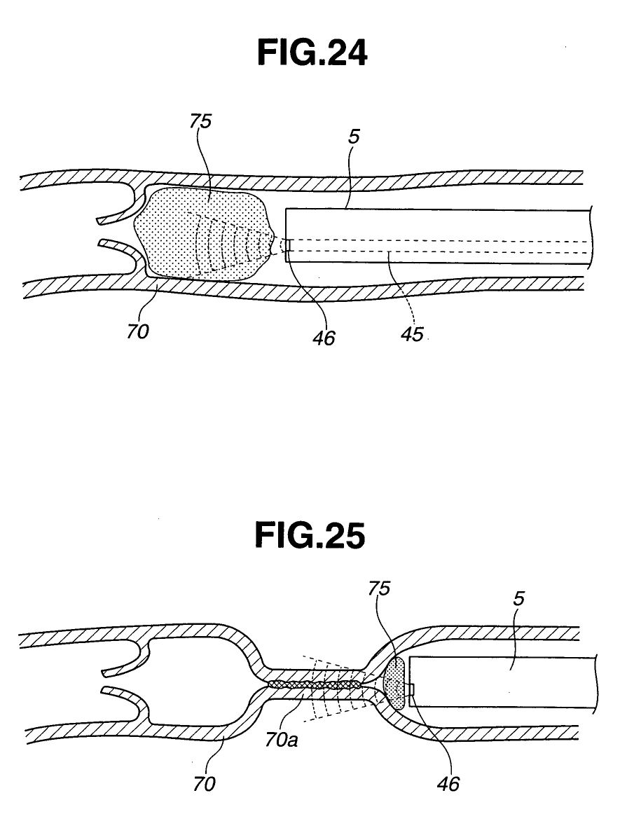

FIGS. 22 to 25 relate to the fourth embodiment of the present invention, wherein: FIG. 22 is a view showing a configuration of a body-lumen-shrinking treatment system; FIG. 23 is a view showing a state where a therapeutic treatment device for body lumen occlusion is inserted into a vein and an indocyanine green (ICG) solution is diffused therein; FIG. 24 is a view showing a state where the indocyanine green (ICG) solution diffused in the vein is irradiated with laser light; and FIG. 25 is a view showing a state where the vein is occluded by the therapeutic treatment device for body lumen occlusion.

As shown in FIG. 22, a body-lumen-shrinking treatment system 1 of the present embodiment includes the suction tube 11 connected with a syringe 51 in stead of the suction device 4. The syringe 51 contains indocyanine green (ICG) solution, which is a laser light absorptive solution. Note that, in the present embodiment, laser light is generated from the laser generating device 41 using a semiconductor laser with a wavelength ranging from not less than 790 nm to not more than 830 nm.

In other words, the body-lumen-shrinking treatment system 1 is constituted to allow ejecting the ICG solution from the suction opening 6 of the first embodiment via the lumen 12, in a state where insides of the lumen 12 of the catheter 2 and the syringe 51 are communicated with each other. That is, the suction opening 6 herein serves as an ejection opening.

With the body-lumen-shrinking treatment system 1 thus constituted, in the therapy for lower-limb varices, the surgeon ejects the ICG solution 75 into the vein 70 after inserting the distal end portion of the catheter main body portion 5 to a position where the vein 70 is to be shrunk and coagulated, as shown in FIG. 23.

Then, like in the third embodiment, the surgeon drives the laser generating device 41 by operating the foot switch or the controller unit so as to irradiate laser light to an area in the vein 70 where the ICG solution 75 was diffused, as shown in FIG. 24.

As a result, the area diffused with the ICG solution 75 absorbs the laser light, and is heated by generating larger amount of heat than other areas. The portion to be treated 70a in the heated area diffused with the ICG solution 75, which includes only the vessel wall of the vein 70, shrinks and coagulates as shown in FIG. 25. The surgeon may further irradiate laser light to the portion to be treated 70a in a state where the portion to be treated 70a of the vein 70 is shrunk, so as to thereby completely occlude the portion to be treated 70a.

Note that, when laser light is irradiated into the vein 70 without diffusing the ICG, it is impossible sufficiently occlude the vein 70 because the heat is dispersed by blood flow.

As described above, the body-lumen-shrinking treatment system 1 of this embodiment can obtain enough amount of heat for shrinking and coagulating a lumen wall by diffusing the ICG solution 75, even if there is blood flow in the vein 70 which is a lumen. Moreover, the present embodiment can not only achieve the effects of the first embodiment, but exclude the need for the suction device 4 leading to an inexpensive constitution, thus allowing for easily occluding a lumen.

Furthermore, although the present embodiment uses the ICG solution since the laser light wavelength ranges from not less than 790 nm to not more than 830 nm, no limitation is placed thereon. Laser light absorptive solutions such as methylene blue, Evans' blue, and crystal violet solutions may also be used suitable for a laser light to be irradiated.

Having described the preferred embodiments of the invention referring to the accompanying drawings, it should be understood that the present invention is not limited to those precise embodiments, and various changes and modifications thereof could be made by one skilled in the art without departing from the spirit or scope of the invention as defined in the appended claims.

What is claimed is:

1. A therapeutic treatment device for body lumen occlusion, comprising:
a pliable tubular main body portion including a fluid path inside thereof, the pliable tubular main body portion being inserted into a body lumen;
an energy generating portion disposed on a side of one end of the main body portion; and
an aperture portion communicating with the fluid path, the aperture portion being disposed in proximity to the energy generating portion.

2. The therapeutic treatment device for body lumen occlusion according to claim 1, wherein the energy generating portion is disposed to a side circumferential portion of the main body portion, and wherein the aperture portion is disposed to the side circumferential surface of the main body portion.

3. The therapeutic treatment device for body lumen occlusion according to claim 1, wherein the energy generating portion is an electrode for discharging a high-frequency current.

4. The therapeutic treatment device for body lumen occlusion according to claim 2, wherein the energy generating portion is an electrode for discharging a high-frequency current.

5. The therapeutic treatment device for body lumen occlusion according to claim 3, wherein the electrode is a pair of bipolar electrodes, and wherein the aperture portion is disposed in a plurality of numbers at approximately the same intervals around the side circumferential portion between the pair of bipolar electrodes.

6. The therapeutic treatment device for body lumen occlusion according to claim 4, wherein the electrode is a pair of bipolar electrodes, and wherein the aperture portion is disposed in a plurality of numbers at approximately the same intervals around the side circumferential portion between the pair of bipolar electrodes.

7. The therapeutic treatment device for body lumen occlusion according to claim 1, wherein the main body portion is provided with an optical fiber for irradiating laser light, and wherein the energy generating portion is disposed on a surface of the one end of the main body portion, and is an irradiation portion for irradiating laser light transmitted by the optical fiber to the body lumen.

8. The therapeutic treatment device for body lumen occlusion according to claim 1, wherein the fluid path is a suction path connected to an external suction device, the suction path being used for negatively pressurizing inside of the body lumen from the aperture portion.

9. The therapeutic treatment device for body lumen occlusion according to claim 2, wherein the fluid path is a suction path connected to an external suction device, the suction path being used for negatively pressurizing inside of the body lumen from the aperture portion.

10. The therapeutic treatment device for body lumen occlusion according to claim 3, wherein the fluid path is a suction path connected to an external suction device, the suction path being used for negatively pressurizing inside of the body lumen from the aperture portion.

11. The therapeutic treatment device for body lumen occlusion according to claim 4, wherein the fluid path is a suction path connected to an external suction device, the suction path being used for negatively pressurizing inside of the body lumen from the aperture portion.

12. The therapeutic treatment device for body lumen occlusion according to claim 5, wherein the fluid path is a suction path connected to an external suction device, the suction path being used for negatively pressurizing inside of the body lumen from the aperture portion.

13. The therapeutic treatment device for body lumen occlusion according to claim 6, wherein the fluid path is a suction path connected to an external suction device, the suction path being used for negatively pressurizing inside of the body lumen from the aperture portion.

14. The therapeutic treatment device for body lumen occlusion according to claim 7, wherein the fluid path is a suction path connected to an external suction device, the suction path being used for negatively pressurizing inside of the body lumen from the aperture portion.

15. The therapeutic treatment device for body lumen occlusion according to claim 7, wherein the fluid path is a solution supplying path used for ejecting a laser light absorptive solution from outside, from the aperture portion into the body lumen.

16. A method for occluding a body lumen using a therapeutic treatment device for body lumen occlusion, the device including: a pliable tubular main body portion including a fluid path inside thereof, the pliable tubular main body portion being inserted into a body lumen; an energy generating portion disposed on a side of one end of the main body portion; and an aperture portion communicating with the fluid path disposed in the main body portion, the aperture portion being disposed in proximity to the energy generating portion,
the method comprising:
inserting the one end of the main body portion where the energy generating portion is disposed into the body lumen to reach a portion to be treated;
shrinking a lumen wall by applying suction to the fluid path to negatively pressurize inside of the body lumen from the aperture portion; and
subjecting a part of the body lumen to heat-occlusion by shrinking and coagulating the part of the body lumen, by applying a predetermined energy to the shrunk lumen wall from the energy generating portion to thereby heat the lumen wall.

17. A method for occluding a body lumen using a therapeutic treatment device for body lumen occlusion, the device including: a pliable tubular main body portion including, inside thereof, a fluid path and an optical fiber, the pliable tubular main body portion being inserted into a body lumen; an irradiation portion for irradiating laser light from the optical fiber, the irradiation portion being disposed on a side of one end of the main body portion; and an aperture portion communicating with the fluid path disposed in the main body portion, the aperture portion being disposed in proximity to the irradiation portion,
the method comprising:
inserting the one end of the main body portion where the energy generating portion is disposed into the body lumen to reach a portion to be treated;
diffusing from the aperture portion a laser light absorptive solution into the body lumen by supplying the laser light absorptive solution to the fluid path; and
subjecting a part of the body lumen to heat-occlusion by shrinking and coagulating the part, by irradiating laser light from the irradiation portion to the laser light absorptive solution to thereby heat the laser light absorptive solution.
